(19) United States
(12) Patent Application Publication
(10) Pub. No.: US 2020/0113980 A1
(43) Pub. Date: Apr. 16, 2020

(54) METHOD OF TREATING AMYOTROPHIC LATERAL SCLEROSIS

(71) Applicants: HUAI-JEN TSAI, Hanbyrg (DE); CHENG-YUNG LIN, New Taipei City (TW)

(72) Inventors: Huai-Jen TSAI, New Taipei City (TW); Cheng-Yung LIN, New Taipei City (TW); Kok-Zhi LEE, New Taipei City (TW); You-Jei CHEN, New Taipei City (TW)

(73) Assignees: HUAI-JEN TSAI, New Taipei City (TW); CHENG-YUNG LIN, New Taipei City (TW)

(21) Appl. No.: 16/676,419

(22) Filed: Nov. 6, 2019

Related U.S. Application Data
(63) Continuation-in-part of application No. 15/604,654, filed on May 25, 2017, now abandoned.

(51) Int. Cl. A61K 38/45; A61P 21/00; A61K 9/00

(52) U.S. Cl. CPC A61K 38/45; A61P 21/00; A61K 45/06; C12Y 207/02003; A61K 9/0019

(57) ABSTRACT

A method of treating an amyotrophic lateral sclerosis in a subject, including administering to the subject suffering from the amyotrophic lateral sclerosis a composition including a therapeutically effective amount of phosphoglycerate kinase.

### CROSS-REFERENCE TO RELATED APPLICATIONS

This application is a Continuation-in-part of the pending U.S. patent application Ser. No. 15/604,654 filed on May 25, 2017, which claims the benefit of Taiwan Patent Application No. 105109607, filed Mar. 28, 2016, all of which are incorporated herein by reference in their entirety. This application also contains a Sequence Listing in computer readable form which is incorporated herein by reference.

### FIELD OF THE INVENTION

The present invention relates to the field of the treatment of amyotrophic lateral sclerosis. More particularly, the present invention relates to a method of treating amyotrophic lateral sclerosis by using phosphoglycerate kinase (PGK), wherein the amyotrophic lateral sclerosis is treated by enhancing the outgrowth of neurites and maintaining neuromuscular junction (NMJ) structure which results in reducing motor neuron denervation.

### DESCRIPTION OF PRIOR ART

The canonical function of phosphoglycerate kinase (PGK) in cytoplasm is to play an important role in the glycolysis pathway that catalyzes the reversible transfer of a phosphate group from 1,3-bisphosphoglycerate (1,3-BPG) to adenosine diphosphate (ADP) and accordingly produces 3-phosphoglycerate (3-PG) and adenosine triphosphate (ATP). In humans, two isozymes of PGK have been identified, PGK1 and PGK2. The isozymes have 87-88% identical amino acid sequence identity; however, they have different localizations. Specifically, it has been reported that PGK1 is ubiquitously expressed in all types of cells, while PGK2 is uniquely expressed in spermatogenic cells.

A neurite refers to the projection from the cell body of a neuron. The neurite can be a dendrite or an axon. In general, the dendrite acts to propagate the electrochemical stimulation received from other neurons to the cell body of the neuron, and the axon conducts the electrochemical stimulation from the cell body to other neurons. Neurodegeneration includes the progressive degeneration or death of neurons, and the impairment of neurites that affects the normal structure or function of neurons (e.g., the transduction of the electrochemical stimulation). It is known that the impairment of neurites may result in amyotrophic lateral sclerosis (ALS), Alzheimer's disease (AD), Parkinson disease (PD) and Huntington's disease (HD). Nevertheless, there is no cure for these neurodegenerative diseases.

In view of the foregoing, there exists in the related art a need for a method for efficiently treating neurodegenerative diseases so as to improve the life quality and/or life span of patients.

### SUMMARY OF THE INVENTION

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the disclosure is directed to a method of enhancing the outgrowth of the neurite of a neuron in a subject. The present method comprises administering to the subject a first therapeutically effective amount of PGK. According to one embodiment of the present disclosure, the PGK is PGK1. According to another embodiment of the present disclosure, the PGK is PGK2.

According to some embodiments of the present disclosure, the neuron is a motor neuron.

According to certain embodiments of the present disclosure, the subject has a neurological disorder. In one specific example, the neurological disorder is a neurodegenerative disease. Non-limiting examples of neurodegenerative diseases include amyotrophic lateral sclerosis (ALS), spinal muscular atrophy (SMA), Alzheimer's disease (AD), Parkinson disease (PD), Huntington's disease (HD), frontotemporal lober dementia (FTLD), Friedreich's ataxia, age-related macular degeneration and Creutzfeldt-Jakob disease.

As would be appreciated, in addition of PGK, an active agent may be co-administered to the subject so as to improve the enhancing and/or therapeutic effect of PGK. Accordingly, the present method may further comprise administering to the subject a second therapeutically effective amount of an active agent. The active agent can be an approved therapeutic agent or an agent in a clinical trial. For example, the active agent may be riluzole, ozanezumab, arimoclomol, tirasemtiv, memantine, dexpramipexole, donepezil, galantamine or rivastigmine.

Another aspect of the present disclosure is directed to a method of treating a neurological disorder in a subject. The method comprises administering to the subject a first therapeutically effective amount of PGK so as to ameliorate or alleviate the symptom associated with the neurological disease.

Optionally, the present method may further comprise administering to the subject a second therapeutically effective amount of an active agent so as to improve the therapeutic effect of PGK. The active agent can be an approved therapeutic agent or an agent in a clinical trial. For example, the active agent may be riluzole, ozanezumab, arimoclomol, tirasemtiv, memantine, dexpramipexole, donepezil, galantamine or rivastigmine.

Aspect of the present disclosure is directed to a method of enhancing the outgrowth of the neurite of a neuron. According to some embodiments of the present disclosure, the method comprises treating the neuron with an effective amount of PGK.

According to certain embodiments of the present disclosure, the neuron is a motor neuron. According to other embodiments of the present disclosure, the effective amount is 1-100 mg/Kg body weight; preferably, 2-80 mg/Kg body weight; more preferably, 5-70 mg/Kg body weight.

The present invention also provides a method of treating an amyotrophic lateral sclerosis (ALS) in a subject, comprising administering to the subject suffering from ALS a composition comprising a therapeutically effective amount of phosphoglycerate kinase.

Many of the attendant features and advantages of the present disclosure will becomes better understood with reference to the following detailed description considered in connection with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A are photographs depicting the morphology of neuronal stem cell NSC-34 administered with specified treatments according to example 1.1 of the present disclosure.

FIG. 1B is a histogram depicting the relative length of neurite of neuronal stem cell NSC-34 administered with specified treatments; the results are analyzed by the software Neurolucida 9.0 according to example 1.1 of the present disclosure.

FIG. 2A are photographs depicting the morphology of neuronal stem cell NSC-34 administered with specified treatments according to example 1.2 of the present disclosure.

FIG. 2B is a histogram depicting the relative length of neurite of neuronal stem cell NSC-34 administered with specified treatments; the results are analyzed by the software Neurolucida 9.0 according to example 1.2 of the present disclosure.

FIG. 3 are fluorescent photographs depicting the length of neurites in the muscles of wild-type strain (WT) and two transgenic lines of zebrafish (Pgk1-knockout and Pgam2-knockout strains). The transgenic lines of zebrafish were generated by the CRISPR/Cas9 construct and microinjection transfer system according to example 2 of the present disclosure.

FIG. 4A are photographs depicting the morphology of neuronal stem cell NSC-34 administered with specified treatments according to example 3 of the present disclosure.

FIG. 4B is a histogram depicting the relative length of neurite of neuronal stem cell NSC-34 administered with specified treatments; the results are analyzed by the software Neurolucida 9.0 according to example 3 of the present disclosure.

FIG. 5 shows that Pgk1 reduces the protein level of p-Cofilin-S3 in the mutated NSC34 (NSC34-SOD1 G93A) and human induced pluripotent stem cells with a point mutation (iPS-SOD1 G85R) cells. FIG. 5A shows NSC34 and NSC34-SOD1 G93A cells cultured in differentiation medium (DM). FIG. 5B shows NSC34 cells cultured in conditional medium (CM) obtained from culturing Sol8 (ATCC® CRL2174™; Mus musculus skeletal muscle) bring NogoA cDNA (So18-NogoA) (lane 1) and So18-NogoA CM plus Pgk1 (lane 2); and NSC34-SOD1 G93A cells cultured in So18-NogoA CM (lane 3) and So18-NogoA CM plus Pgk1 (lane 4). When Pgk1 is added in So18-NogoA CM to culture NSC34-SOD1 G93A cells, the amount of p-Cofilin-S3 is reduced. FIG. 5C shows protein levels of p-Limk1-508, -S323, Limk1, p-Cofilin-S3 and Cofilin of human iPS-SOD1 G85R cells cultured in medium containing Pgk1 at concentration of 33- and 66-ng/ml. When Pgk1 is added in culture medium containing human diseased motor neuron cells iPS-SOD1 G85R, the amount of p-Limk-S323 and p-Cofilin-S3 are also reduced. Protein level of α-tubulin serves as internal control. Scale bar: 25 μm.

FIG. 6 shows that addition of Pgk1 rescues denervation of amyotrophic lateral sclerosis (ALS) mouse model. The neuromuscular junction (NMJ) phenotype of ALS mouse model harboring human SOD1-G93A after intramuscular injection of Phosphate buffered saline (PBS) (FIGS. 6A, 6B and 6C) and Pgk1 (FIGS. 6D, 6E and 6F) in the gastrocnemius muscle of the right hind leg. Neurofilament-H (NF-H) and SV2 labeled by green fluorescent signal are used to detect the axon terminal of motoneurons, while α-BTX labeled by red fluorescence signal is used to detect the acetylcholine receptor on motor endplates. FIG. 6G shows statistical analysis of the number of innervated NMJ among PBS-injected WT mouse, PBS-injected SOD1-G93A mouse and Pgk1-injected SOD1-G93A mouse. Statistical analysis used Student's t-test (***, significant difference at p<0.001).

FIG. 7 shows the effect of Pgk1 injection on the exercise capability of hind leg of ALS mice. PBS (SOD1 G93A/PBS) or Pgk1 (SOD1 G93A/Pgk1) is injected into the gastrocnemius muscle of the right hind leg of 60-day-old transgenic SOD1 G93A mice, respectively. Continuous injection is carried out every 15 days until mice are 120 days old. FIG. 7A shows that the frequency of muscle contraction of both hind legs is counted individually when the mice are 130 days old. FIG. 7B shows that the exercise capability value of each mouse is calculated from the increased fold(s) in the number of contractions of injected (right) leg versus that of uninjected (left) leg. Data of each group are averaged from three mice and presented as mean±S.D. Student's t-test is used for statistical analysis (**, significant difference at p<0.01).

In accordance with common practice, the various described features/elements are not drawn to scale but instead are drawn to best illustrate specific features/elements relevant to the present invention.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

As used herein, the term “neuron” refers to an animal cell comprising a cell body and a plurality of neurites respectively extruded from the cell body, in which the neurites include axons and dendrites. Exemplary neurons include sensory neurons, motor neurons and interneurons. In addition, the term “neuron” as used herein also refers to neurosustentacular cells, glia cells, Schwann cells and the neurons constituting a central nervous system, a brain, a brain stem, a spinal cord and synaptic regions of the central nervous system and peripheral nervous systems.

As used herein, the term “neurite outgrowth” refers to the process of axons or dendrites growing out of the cell body of a neuron. In general, the neurite outgrowth plays a key role in the development and regeneration of neurons. The neurite outgrowth improves the neural connectivity thereby promoting the synapse formation or remodeling the synapses.

The term “neural connectivity” as used herein refers to the number, type, and quality of connections (i.e., synapses) between neurons in an organism. Synapses form between neurons, between neurons and muscles (i.e., neuromuscular junction), and between neurons and other biological structures, including internal organs, endocrine glands, and the like. Synapses are specialized structures, by which neurons transmit chemical or electrical signals to each other and to non-neuronal cells, muscles, tissues, and organs. Compounds that affect neural connectivity may do so by establishing new synapses (e.g., by neurite outgrowth or neurite activation) or by altering or remodeling existing synapses. Synaptic remodeling refers to changes in the quality, intensity or type of signal transmitted at particular synapses.

As used herein, the term “neurological disorder” refers to a neuropathy, a neurodegenerative disease and/or other neuron-associated diseases caused by mechanical damage (e.g., trauma), chemical damage (e.g., neurotoxin, immunosuppression resulting from the treatment or side-effect), or viral infection. Exemplary neurodegenerative diseases include, but are not limited to, amyotrophic lateral sclerosis (ALS), spinal muscular atrophy (SMA), Alzheimer's disease (AD), Parkinson disease (PD), Huntington's disease (HD), frontotemporal lober dementia (FTLD), Friedreich's ataxia, age-related macular degeneration and Creutzfeldt-Jakob disease. The neuron-associated disease may include ocular disease caused by neuronal damage (e.g., glaucoma), Bell's palsy, or other forms of localized paralysis, neuron based impotence, such as caused by nerve trauma following radical prostatectomy, or other complaints. In the present disclosure, the term “neurological disorder” can be any of central nervous system (CNS) disease, peripheral nervous system (PNS) disease, sympathetic nervous system disease or parasympathetic nervous system disease.

The term “treating” or “treatment” encompasses partially or completely preventing, ameliorating, mitigating and/or managing a symptom, a secondary disorder or a condition. Preferably, the term “treating” or “treatment” as used herein refers to application or administration of the present PGK or the pharmaceutically acceptable salts thereof to a subject, who has a symptom, a secondary disorder or a condition associated with neurological disorders, with the purpose to partially or completely alleviate, ameliorate, relieve, delay onset of, inhibit progression of, reduce severity of, and/or reduce incidence of one or more symptoms, secondary disorders or features associated with the neurological disorders.

The term “effective amount” as referred to herein designate the quantity of a component which is sufficient to yield a desired response. For therapeutic purposes, the effective amount is also one in which any toxic or detrimental effects of the component are outweighed by the therapeutically beneficial effects. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives. Effective amount may be expressed, for example, in grams, milligrams or micrograms or as milligrams per kilogram of body weight (mg/Kg). Persons having ordinary skills could calculate the human equivalent dose (HED) for the medicament (such as the present PGK) based on the doses determined from animal models. For example, one may follow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

Unless otherwise indicated, a “therapeutically effective amount” of the present PGK is an amount sufficient to provide a therapeutic benefit in the treatment or management of a disease or condition, or to delay or minimize one or more symptoms associated with the disease or condition. A therapeutically effective amount of the present PGK is an amount of therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of a disease or condition, or enhances the therapeutic efficacy of another therapeutic agent.

The term “subject” refers to a mammal including the human species that is treatable with methods of the present invention. The term “subject” is intended to refer to both the male and female gender unless one gender is specifically indicated.

The present disclosure is based, at least in part, on the discovery that PGK is useful in enhancing the neurite outgrowth of motoneurons, reducing NMJ denervation and accordingly, providing a potential means to treat neurological disorders, especially the neurodegenerative diseases.

The first aspect of the present disclosure is directed to a method of in vitro enhancing the neurite outgrowth of a neuron; the method comprises treating the neuron with an effective amount of PGK. According to one embodiment of the present disclosure, the PGK is PGK1 and has the amino acid sequence of SEQ ID NO: 1. According to another embodiment of the present disclosure, the PGK is PGK2 and has the amino acid sequence of SEQ ID NO: 2.

According to the embodiments of the present disclosure, the neuron may be a sensory neuron, a motor neuron or an interneuron. In one specific example, the neuron is a motor neuron.

According to some embodiments of the present disclosure, the effective amount of PGK is about 1-100 mg/Kg body weight; preferably, about 2-80 mg/Kg body weight; more preferably 5-70 mg/Kg body weight.

The second aspect of the present disclosure is directed to a method of enhancing the outgrowth of the neurite of a neuron in a subject. According to certain embodiments, the present method comprises administering to the subject a therapeutically effective amount of PGK. According to one embodiment of the present disclosure, the PGK is PGK1 and has the amino acid sequence of SEQ ID NO: 1. According to another embodiment of the present disclosure, the PGK is PGK2 and has the amino acid sequence of SEQ ID NO: 2.

According to some embodiments, the subject has a neurological disorder. In these embodiments, the neurological disorder is caused by the impairment of neurites; for example, the subject may suffer from a neuronal damage or neuropathy caused by a mechanical damage (e.g., trauma), a chemical damage (e.g., neurotoxin, immunosuppression resulting from the treatment or the side-effect of the treatment), and/or a biological damage (e.g., viral infection, autoimmune, aging, disease, metabolic disorder or abnormal expression of protein) that interferes the transduction of electrochemical stimulation in the subject. The present method is useful in enhancing the outgrowth of the neurites in the subject so as to treat the neurological disease.

In one specific example, the subject has a neurodegenerative disease. Exemplary neurodegenerative diseases include, but are not limited to amyotrophic lateral sclerosis, spinal muscular atrophy, Alzheimer's disease, Parkinson disease, Huntington's disease, frontotemporal lober dementia, Friedreich's ataxia, age-related macular degeneration and Creutzfeldt-Jakob disease.

Depending on the particular condition (e.g., the physical condition of the patient, the type of neurological disorder, and the severity of the neurological disorder), the therapeutically effective amount of PGK may be divided into one or more doses throughout a designated time period so as to enhance the outgrowth of the neurites and/or treat the neurological disorder in the subject; for example, the effective amount of PGK may be divided into 1, 2, 3, 4, 5, 6, 7, 8, 9, 10 or more doses. The time period between two consecutive doses may be 1, 2, 3, 4, 5, 6, 7, 8, 9, 10 or more days. The practitioner may adjust the administration procedure in accordance with the desired effects.

The present PGK is useful in enhancing the outgrowth of the neurites thereby treating the neurological disorder in a subject. As would be appreciated, a suitable active agent may be co-administered with the PGK to the subject (i.e., be administered to the subject before, concurrent with, or after the administration of PGK) for the purpose of enhancing the therapeutic effect. Accordingly, the present method may further comprises administering to the subject a therapeutically effective amount of an active agent. In general, the active agent may be an approved therapeutic agent or an agent in a clinical trial that has a beneficial effect on the neurological disorders (e.g., the neurodegenerative diseases). For example, the active agent may be riluzole, ozanezumab, arimoclomol, tirasemtiv, memantine, dexpramipexole, donepezil, galantamine or rivastigmine.

Depending on desired effect, the present PGK may be administered to the subject before, concurrent with, or after the administration of the active agent. In addition, the effective amount of PGK/active agent may be divided into one or more doses throughout a designated time period; for example, the effective amount of PGK/active agent may be divided into 1, 2, 3, 4, 5, 6, 7, 8, 9, 10 or more doses. The time period between two consecutive doses of the PGK/active agent may be 1, 2, 3, 4, 5, 6, 7, 8, 9, 10 or more days. The practitioner may adjust the administration procedure in accordance with the desired effects.

According to one embodiment of the present disclosure, the subject is a mammal, including a human, a mouse and a rat.

The third aspect of the present disclosure pertains to a pharmaceutical composition that comprises a first effective amount of PGK and a second effective amount of an active agent. According to the embodiments, the PGK is PGK1 or PGK2. Non-limiting examples of the active agent include, riluzole, ozanezumab, arimoclomol, tirasemtiv, memantine, dexpramipexole, donepezil, galantamine and rivastigmine.

The present invention further provides a method of treating an amyotrophic lateral sclerosis (ALS) in a subject, comprising administering to the subject suffering from ALS a composition comprising a therapeutically effective amount of phosphoglycerate kinase (PGK). (Please see C. Y. Lin et al., Extracellular Pgk1 enhances neurite outgrowth of motoneurons through Nogo66/NgR-independent targeting of NogoA, eLife 2019; 8:e49175. DOI: https://doi.org/10.7554/eLife.49175, which is incorporated herein by reference in its entirety.)

In one embodiment, the subject is an animal, preferably a mammal, more preferably a human.

In another embodiment, the PGK is PGK1 or PKG2. In a preferred embodiment, the PGK is PGK1.

In one embodiment, the PGK1 comprises the amino acid sequence of SEQ ID NO: 1. In another embodiment, the PGK2 comprises the amino acid sequence of SEQ ID NO: 2.

In one embodiment, the amyotrophic lateral sclerosis is treated by enhancing the outgrowth of the neurites of impaired neurons in the subject. In a preferred embodiment, the impaired neurons have impaired neurites.

In another embodiment, the neurons are motor neurons. In a preferred embodiment, the amyotrophic lateral sclerosis is treated by enhancing the innervation of neuromuscular junction (NMJ) in the subject. In a more preferred embodiment, the amyotrophic lateral sclerosis is treated by maintaining normal motor neurons to innervate muscle contraction in the subject.

In one embodiment, the composition further comprises a therapeutically effective amount of an active agent. In a preferred embodiment, the active agent is riluzole, ozanezumab, arimoclomol, tirasemtiv, memantine, dexpramipexole, donepezil, galantamine or rivastigmine.

The composition of the present invention further comprises a pharmaceutically acceptable carrier which may be administered to a subject through a number of different routes known in the art. In one embodiment, the composition with a pharmaceutically acceptable carrier are administered externally, intraperitoneally, intravenously, subcutaneously, topically, orally; or by intrathecal Baclofen, muscle or inhalation. The composition will be delivered to target sites by the digestive system or the circulatory system. In a preferred embodiment, the administration of the composition is intramuscular administration.

As used herein, the term “pharmaceutically acceptable carrier” is determined in part by the particular composition being administered, as well as by the particular method used to administer the composition. The carrier may include aqueous isotonic sterile injection solutions, which may contain antioxidants, buffers, bacteriostatic agents, and solutes that render the formulation isotonic with the blood of the intended recipient, and aqueous and non-aqueous sterile suspensions that can include suspending agents, solubilizers, thickening agents, stabilizers, preservatives, liposomes, microspheres and emulsions. The term “pharmaceutically acceptable carrier” refers to compounds and compositions which can be administered to mammals without undue toxicity.

The composition and the pharmaceutically acceptable carrier may be formulated via sterile aqueous solutions or dispersions, aqueous suspensions, oil emulsions, water in oil-in-oil emulsions, specific emulsification liquids, long residence emulsifiers, viscous emulsions, microemulsions, nanoemulsions, liposomes, microparticles, microspheres, nanospheres, nanoparticles, micromercury, and several sustainable releases of natural or synthetic polymers.

In another embodiment, the therapeutically effective amount of PGK ranges from 1 to 100 mg/Kg body weight. In a preferred embodiment, the therapeutically effective amount of PGK ranges from 2 to 80 mg/Kg body weight. In a more preferred embodiment, the therapeutically effective amount of PGK ranges from 5 to 70 mg/Kg body weight.

In one embodiment, the therapeutically effective amount of PGK1 ranges from 1 to 100 mg/Kg body weight. In a preferred embodiment, the therapeutically effective amount of PGK1 ranges from 2 to 80 mg/Kg body weight. In a more preferred embodiment, the therapeutically effective amount of PGK1 ranges from 5 to 70 mg/Kg body weight.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLES

The present invention may be implemented in many different forms and should not be construed as limited to the examples set forth herein. The described examples are not limited to the scope of the present invention as described in the claims.

Materials and Methods

Culture and Differentiation of Sol8 Cells

The Sol8 (ATCC, CRL-2174) cells were cultured and differentiated in high glucose DMEM medium containing 10% heat-inactivated fetal bovine serum (FBS), 1% penicillin and 1% streptomycin; two days later, 1 μg/μl of doxycycline was added to the medium so as to induce the expression of target genes. The culture medium was collected and the fresh medium containing doxycycline was added to the Sol8 cells every 24 hours. The collected culture medium was then centrifuged at a speed of 1,000 rpm for 5 minutes at room temperature, the supernatant harvested therefrom served as the conditioned medium for the following assays.

Culture and Differentiation of NSC34 Cells

The NSC34 cells (Cedarlane, CLU-140) were cultured in high glucose DMEM medium containing 10% heat-inactivated FBS, 1% penicillin and 1% streptomycin. To analyze the effect of PGK, the medium was replaced with the differentiation medium (high glucose DMEM medium containing 2.5% heat-inactivated FBS, 1% penicillin and 1% streptomycin). Two days later, the differentiation medium was replaced with the conditioned medium collected from the Sol8 cell culture. The conditioned medium was replaced every 24 hours, and the outgrowth of neurites was evaluated by microscopy.

Evaluation of Neurite Outgrowth by Use of Anti-PGK1 Antibody

The NSC34 cells were cultured in the differentiation medium (DM) (high glucose DMEM medium containing 2.5% heat-inactivated FBS, 1% penicillin and 1% streptomycin). Two days later, the differentiation medium was replaced with the conditioned medium containing anti-PGK1 antibody (final concentration: 1 μg/ml, abcam, ab38007). The antibody-containing conditioned medium was replaced every 24 hours, and the outgrowth of neurites was evaluated by microscopy.

Evaluation of Neurite Outgrowth by Use of PGK1 or PGK2 Polypeptide

The NSC34 cells were cultured in the differentiation medium (DM) (high glucose DMEM medium with 2.5% heat-inactivated FBS, 1% penicillin and 1% streptomycin) containing the PGK1 or PGK2 polypeptide (final concentration: 33 μg/ml). The differentiation medium containing the PGK1 or PGK2 polypeptide was replaced every 24 hours, and the outgrowth of neurites was evaluated by microscopy.

Transgenic Zebrafish

The wild-type strain and the transgenic lines of zebrafish, Tg (mnx1:EGFP) were purchased from the Zebrafish International Resource Center (ZIRC, USA).

The microinjection materials, including RNA (Pgk1 sgRNA) and DNA (pZa-Cas9), were diluted by distilled water. The 2.3 nL of the microinjection materials was microinjected into the animal pole of blastula stage zebrafish embryos, in which microinjection materials comprised 0.0575 ng of pZa-Cas9 (the concentration of pZa-Cas9 was 25 ng/μl) and 0.0125 ng of Pgk1 sgRNA. After the injection of pZa-Cas9, the Cas9-2A-tRFP driven by actin promoter was expressed in muscles; the encoded Cas9 then interacted with Pgk1 sgRNA to inhibit the expression of Pgk1 in muscles.

The wild-type strain and Tg (mnx1:EGFP) respectively administered with specified treatments were monitored by anatomic microscope (MZFLIII, Leica) equipped with fluorescence system (Hg 100w filter set GFP-Plus) and DFC 490 (Leica) CCD digital camera. The images were taken and analyzed by ACDsee software. The fluorescent images of fish embryos and cryosections were analyzed by Zeiss LSM 780 Confocal microscope equipped with zen 2009 light edition software.

### Example 1 Effect of PGK1 on Neurite Outgrowth

The effects of the anti-PGK1 antibody and the PGK1 polypeptide on the neurite outgrowth were respectively examined in example 1.1 and example 1.2.

### Example 1.1 Inhibitory Effect of Anti-PGK1 Antibody

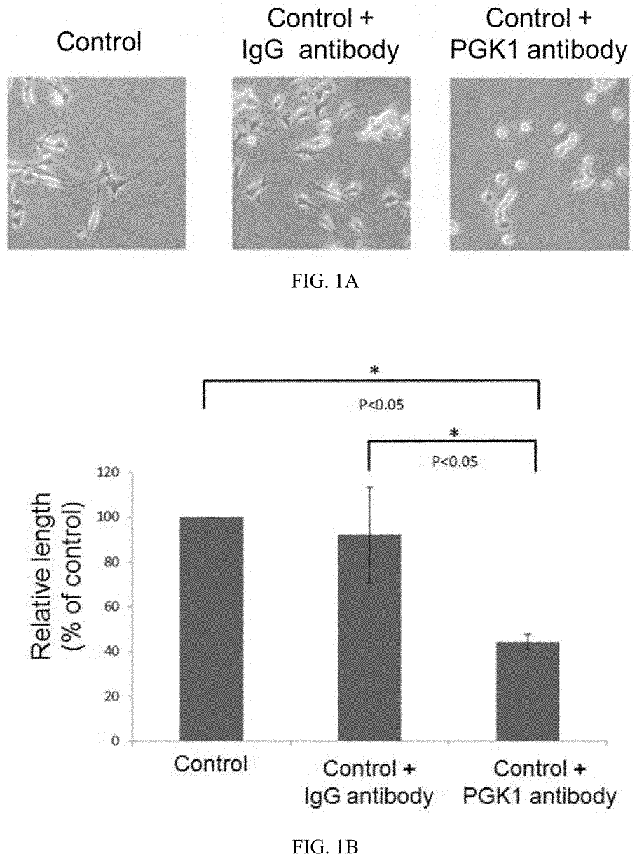

The NSC-34 cells were respectively treated with (i) Sol8 conditioned medium (So18-CM) (control group), (ii) Sol8 conditioned medium containing anti-IgG antibody (control group+IgG antibody; serving as the antibody control group), and (iii) So18 conditioned medium containing anti-PGK1 antibody (control group+PGK1 antibody; for the purpose of blocking the signal pathway of PGK1). The results were respectively illustrated in FIGS. 1A and 1B.

FIG. 1A illustrated the morphology of cells respectively treated with specified treatments, and FIG. 1B depicted the analysis result. According to the data, compared with the control group, anti-IgG antibody had no effect on the neurite outgrowth, while the anti-PGK1 antibody significantly inhibited the outgrowth of neurites.

These data indicated that the blockage of PGK1 signal pathway by use of the anti-PGK1 antibody significantly inhibited the outgrowth of neurites.

### Example 1.2 Treatment of PKG1 Polypeptide

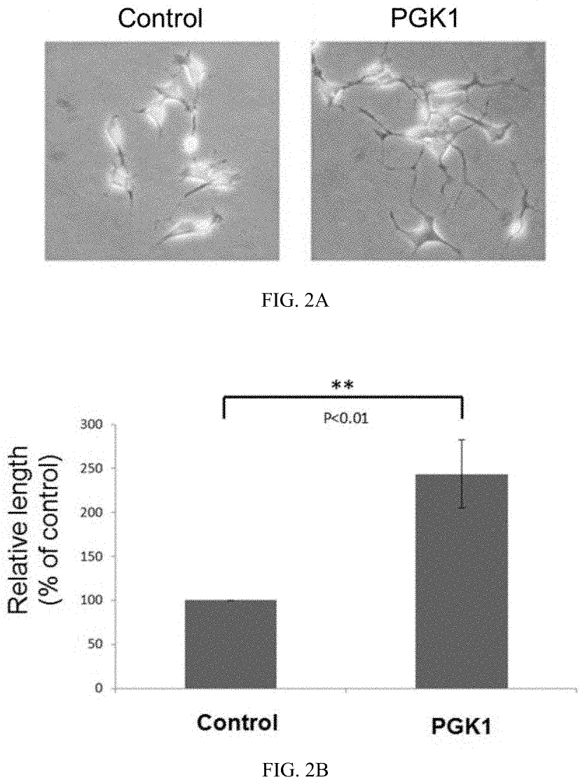

To further confirm the effect of PGK1 on the neurite outgrowth, the NSC-34 motor neurons were then respectively treated with (i) DMEM containing 2.5% FBS (control group), and (ii) DMEM containing 2.5% FBS and PGK1 polypeptide (PGK1 group). In general, the DMEM medium containing 2.5% FBS provides a minimum support for the growth of neurons, and accordingly, the effect of tested agent (e.g., PGK1 polypeptide) on neurite could be thoroughly investigated in the environment. The results were respectively illustrated in FIGS. 2A and 2B.

FIG. 2A illustrated the morphology of cells respectively treated with specified treatments, and FIG. 2B depicted the analysis result. The data indicated that compared with the control group, the treatment of PGK1 polypeptide significantly enhanced the outgrowth of neurites.

### Example 2 In Vivo Effect of PGK1 on Neurite Outgrowth

The zebrafish model was used in this example to examine the in vivo effect of PGK1 on neurite outgrowth, in which the expression of PGK1 in PGK1 KO (the PGK1 gene was knockout) transgenic zebrafish was suppressed by the CRISPER/Cas9 system, and the Pgam2 KO transgenic zebrafish (the Pgam2 gene was knockout) served as the knockout control group.

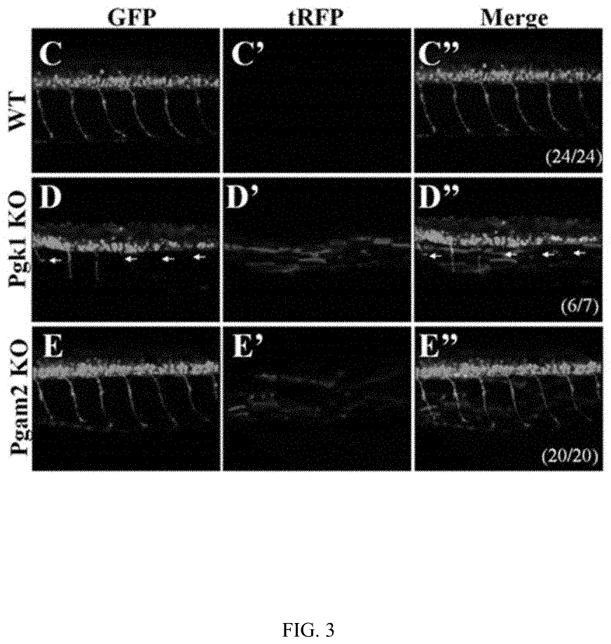

The images of FIG. 3 illustrated that PGK1 knockout significantly reduced the length of neurite in the muscle of PGK1 KO transgenic zebrafish as compared to the wild-type strain and the Pgam2 KO transgenic zebrafish. These results demonstrated that PGK1 plays an important role in the in vivo outgrowth process of neurites.

### Example 3 Effect of PGK2 on Neurite Outgrowth

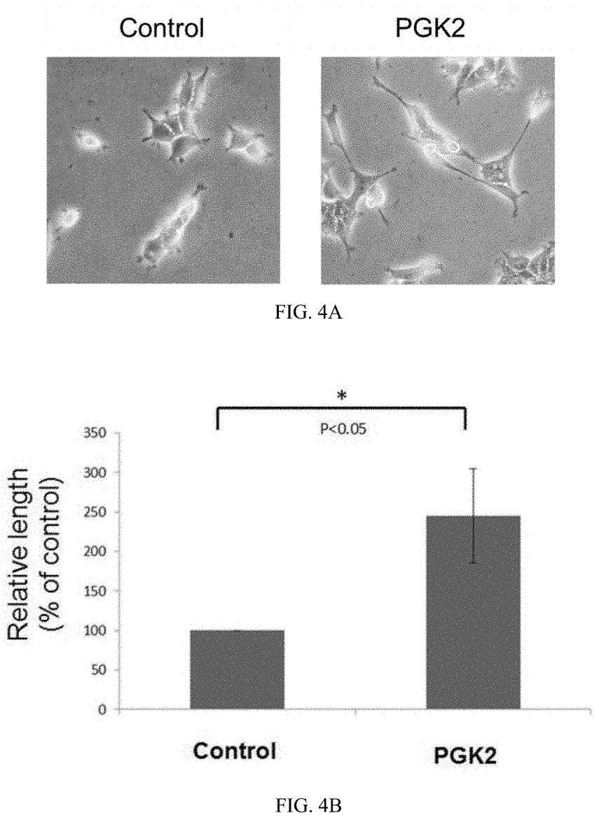

In addition to PGK1, the invention further examined the effect of PGK2 on the neurite outgrowth. The NSC-34 motor neurons were respectively treated with (i) DMEM containing 2.5% FBS (control group), and (ii) DMEM containing 2.5% FBS and PGK2 protein (PGK2 group). The results were respectively illustrated in FIGS. 4A and 4B.

FIG. 4A illustrated the morphology of cells respectively treated with specified treatments, and FIG. 4B depicted the analysis result. As the data of PGK1, compared with the control group, the treatment of PGK2 protein significantly enhanced the neurite outgrowth.

In conclusion, the present disclosure demonstrates that the treatment of PGK1 or PGK2 significantly enhances the outgrowth of neurites. Based on the impairment of neurites generally results in neurological disorders, the present disclosure provides a potential means to treat the neurological disorders by use of PGK1 and/or PGK2.

### Example 4 the Signaling Pathway Underlying the Involvement of Extracellular Pgk1-Mediated Reduction of p-Cofilin-S3 in ALS (SOD1 G85R) Cell Line

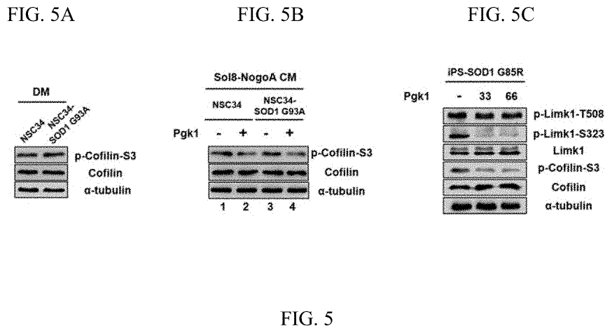

The present invention used mouse motor neuron cell lines (NSC34, NSC34-SOD1 G93A), human ALS cell line (iPS-SOD1 G85R) and mouse ALS model to determine the beneficial effects of Pgk1 supplementation. To accomplish this, the present invention performed an in vitro study, in which NSC34 cells overexpressing the ALS-associated mutant SOD1 G93A (NSC34-SOD1 G93A) were cultured in DM and So18-NogoA CM. When cultured in DM, the protein level of endogenous p-Cofilin-S3 exhibited no difference between NSC34 and NSC34-SOD1 G93A cells (FIG. 5A). However, when cultured in So18-NogoA condition medium (CM), the amount of p-Cofilin-S3 in NSC34 cells was sharply increased compared to that of NSC34 cultured in Sol8 CM. Meanwhile, when Pgk1 was added in So18-NogoA CM to culture NSC34-SOD1 G93A cells, the amount of p-Cofilin-S3 was reduced (FIG. 5B). The present invention differentiated motor neurons derived from human induced pluripotent stem cells (iPSCs) harboring human SOD1 mutant (G85R) and then added Pgk1. Pgk1 addition reduced p-Cofilin through decreasing p-Limk1-5323 (FIG. 5C), as noted above, indicating that extracellular Pgk1 induced the same signal transduction pathway as that determined from the mouse NSC34 cell line and human motor neurons.

### Example 5 Intramuscular Injection of Pgk1 was Able to Rescue Neuromuscular Junction (NMJ) Denervation of ALS Model (SOD1 G93A) Transgenic Mice

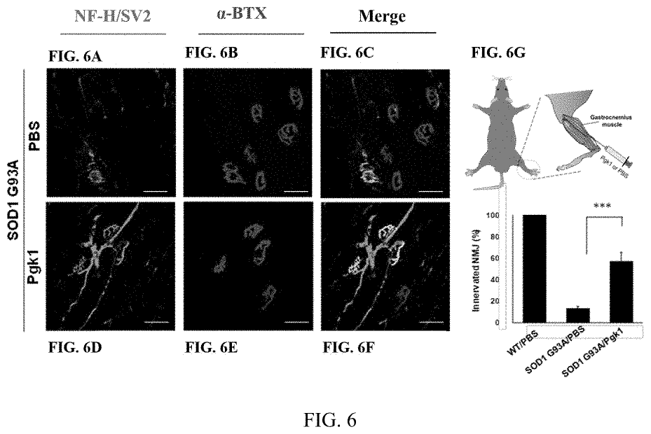

The present invention performed intramuscular injection of Pgk1 (30 μg) into the gastrocnemius muscle of the right hind leg of 60-day-old transgenic SOD1 G93A mice, followed by another injection every 15 days until mice were 120 days old. The present invention quantified the proportion of innervated neuromuscular junction (NMJ) in the gastrocnemius muscle of the right hind leg of mice. Compared to the control group, the proportion of innervated NMJ of wild type (WT) mice injected with PBS (WT/PBS), which was normalized as 100%, the proportion of innervated NMJ of SOD1 G93A/PBS was 13±0.02%, indicating that the signal of motor neuron axon and axon terminal (NF-H/SV2) was significantly reduced. However, the proportion of innervated NMJ of SOD1 G93A mice injected with Pgk1 (SOD1 G93A/Pgk1) was 57±0.08%, indicating that supplementary addition of Pgk1 could enhance NMJ innervation (FIG. 6).

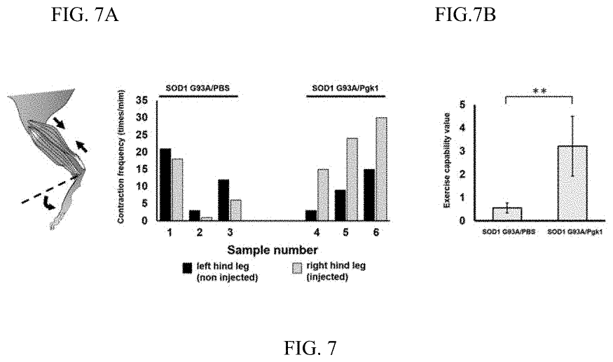

Furthermore, the present invention examined the muscle contraction ability of hind leg in 130-day-old mice. In the WT/PBS group, the muscle contraction of both hind legs was normal, exhibiting strong movement. In contrast, in the SOD1 G93A/PBS group, muscle contraction of both hind legs was extremely poor (FIG. 7). Nevertheless, in the SOD1 G93A/Pgk1 group, muscle contraction of the left hind leg is as poor as that of the SOD1 G93A/PBS mice. Interestingly, muscle contraction of Pgk1-injected right hind leg remained functional, exhibiting a superior movement. Exercise capability value of the SOD1 G93A/Pgk1 group was significantly higher than that of the SOD1 G93A/PBS group (FIG. 7). Additionally, the present invention found that the proportion of innervated NMJ in the gastrocnemius muscle of the Pgk1-injected right hind leg was increased compared to that of the left hind leg. Taken together, it indicated that the Pgk1-injected right hind leg of SOD1 G93A mice maintained some normal motor neurons to innervate muscle contraction.

In addition, the experiments and the data in C. Y. Lin et al., (Extracellular Pgk1 enhances neurite outgrowth of motoneurons through Nogo66/NgR-independent targeting of NogoA, eLife 2019; 8:e49175) were also incorporated herein by reference in its entirety.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method of treating an amyotrophic lateral sclerosis (ALS) in a subject, comprising administering to the subject suffering from ALS a composition comprising a therapeutically effective amount of phosphoglycerate kinase (PGK).

2. The method of claim 1, wherein the PGK is PGK1 or PGK2.

3. The method of claim 1, wherein the subject is a human.

4. The method of claim 1, wherein the effective amount of PGK is sufficient to treat the ALS by enhancing the outgrowth of the neurites of impaired neurons in the subject.

5. The method of claim 4, wherein the neurons are motor neurons.

6. The method of claim 1, wherein the effective amount of PGK is sufficient to treat the ALS by maintaining the innervation of neuromuscular junction in the subject.

7. The method of claim 1, wherein the composition further comprises a therapeutically effective amount of an active agent.

8. The method of claim 7, wherein the active agent is riluzole, ozanezumab, arimoclomol, tirasemtiv, memantine, dexpramipexole, donepezil, galantamine or rivastigmine.

9. The method of claim 1, wherein the administration of the composition is intramuscular administration.

10. The method of claim 1, wherein the therapeutically effective amount of PGK ranges from 1 to 100 mg/Kg body weight.

11. The method of claim 1, wherein the therapeutically effective amount of PGK ranges from 2 to 80 mg/Kg body weight.


SEQUENCE LISTING (2 sequences)

SEQ ID NO: 1 - protein, 417 aa, Artificial Sequence
  PGK1
MSLSNKLTLDKLDVKGKRVVMRVDFNVPMKNNQITNNQRIKAAVPSIKFCLDNGAKSVVLMSHLGRPDGVPMPDKYSLEPVAVELKSLLGKDVLFLKDCVGPEVEKACANPAAGSVILLENLRFHVEEEGKGKDASGNKVKAEPAKIEAFRASLSKLGDVYVNDAFGTAHRAHSSMVGVNLPQKAGGFLMKKELNYFAKALESPERPFLAILGGAKVADKIQLINNMLDKVNEMIIGGGMAFTFLKVLNNMEIGTSLFDEEGAKIVKDLMSKAEKNGVKITLPVDFVTADKFDENAKTGQATVASGIPAGWMGLDCGPESSKKYAEAVTRAKQIVWNGPVGVFEWEAFARGTKALMDEVVKATSRGCITIIGGGDTATCCAKWNTEDKVSHVSTGGGASLELLEGKVLPGVDALSNI

SEQ ID NO: 2 - protein, 417 aa, Artificial Sequence
  PGK2
MSLSKKLTLDKLDVRGKRVIMRVDFNVPMKKNQITNNQRIKASIPSIKYCLDNGAKAVVLMSHLGRPDGVPMPDKYSLAPVAVELKSLLGKDVLFLKDCVGAEVEKACANPAPGSVILLENLRFHVEEEGKGQDPSGKKIKAEPDKIEAFRASLSKLGDVYVNDAFGTAHRAHSSMVGVNLPHKASGFLMKKELDYFAKALENPVRPFLAILGGAKVADKIQLIKNMLDKVNEMIIGGGMAYTFLKVLNNMEIGASLFDEEGAKIVKDIMAKAQKNGVRITFPVDFVTGDKFDENAQVGKATVASGISPGWMGLDCGPESNKNHAQVVAQARLIVWNGPLGVFEWDAFAKGTKALMDEIVKATSKGCITVIGGGDTATCCAKWNTEDKVSHVSTGGGASLELLEGKILPGVEALSNM